(19) United States
(12) Plant Patent Application Publication
(10) Pub. No.: US 2015/0373894 P1
(43) Pub. Date: Dec. 24, 2015

(54) Liriope muscari plant named 'VS001'

(50) ;

(71) Applicant: Todd Anthony Layt, Clarendon (AU)

(72) Inventor: Todd Anthony Layt, Clarendon (AU)

(21) Appl. No.: 14/120,720

(22) Filed: Jun. 19, 2014

(51) Int. Cl. A01H 5/00

(52) U.S. Cl. PLT/450

(57) ABSTRACT

‘VS001’ is a distinctive variety of Liriope muscari which is characterized by foliage variegation ranging from green to lime-green striations in juvenile shoots and immature foliage, to green and pale yellow to white striations in mature foliage and varying amounts of each color at intermediate stages of leaf development.

### LATIN NAME OF THE GENUS AND SPECIES

The Latin name of the genus and species of the novel variety disclosed herein is Liriope muscari.

### VARIETY DENOMINATION

The inventive variety of Liriope muscari disclosed herein has been given the variety denomination ‘VS001’.

### BACKGROUND OF THE INVENTION

The present invention relates to a new and distinct perennial variety of Liriope muscari, which has been given the variety denomination of ‘VS001’. Its market class is that of an ornamental flowering perennial. ‘VS001’ is intended for use in landscaping and also in garden containers. Liriope muscari, commonly called “lilyturf”, is an evergreen perennial native to China and Japan that forms clumps of dark green grass-like leaves with light lavender flower spikes that appear in summer. Its consistent size and performance through its hardiness range make it an ideal choice for specimen, borders and mass plantings in any filtered sun to shady, low-maintenance landscape or container.

Parentage: In March 2004, a variegated mutation was discovered growing from a Liriope muscari ‘samantha’ (unpatented) stock plant at a wholesale nursery in Richmond, New South Wales, Australia. The instant mutation was divided from the mother plant, a non-variegated form of Liriope muscari, and potted into a nursery container for further observation. Said plant was further grown and asexually propagated through four generations from August 2004 until January 2011, at which time it was determined that the characteristics for which the instant mutation was originally isolated were uniform and stable. The new plant was given the name ‘VS001’.

Asexual Reproduction: ‘VS001’ was first asexually propagated by dividing the root-bearing, rhizomatous propagules of the plant (i.e. “division cloning”) in August of 2004 at a wholesale nursery in Richmond, New South Wales, Australia and has since been asexually propagated through four subsequent generations. The distinctive characteristics of the inventive ‘VS001’ variety are stable from generation to generation; clones of the variety produced by asexual reproduction maintain the distinguishing characteristics of the original plant.

### SUMMARY OF THE INVENTION

‘VS001’ is a distinctive variety of Liriope muscari which is characterized by foliage variegation ranging from green to lime-green striations in juvenile shoots and immature foliage, to green and pale yellow to white striations in mature foliage and varying amounts of each color at intermediate stages of leaf development.

### BRIEF DESCRIPTION OF THE FIGURES

FIG. 1 shows an exemplary ‘VS001’ specimen, approximately 8 months of age, grown in 15 cm nursery pots.

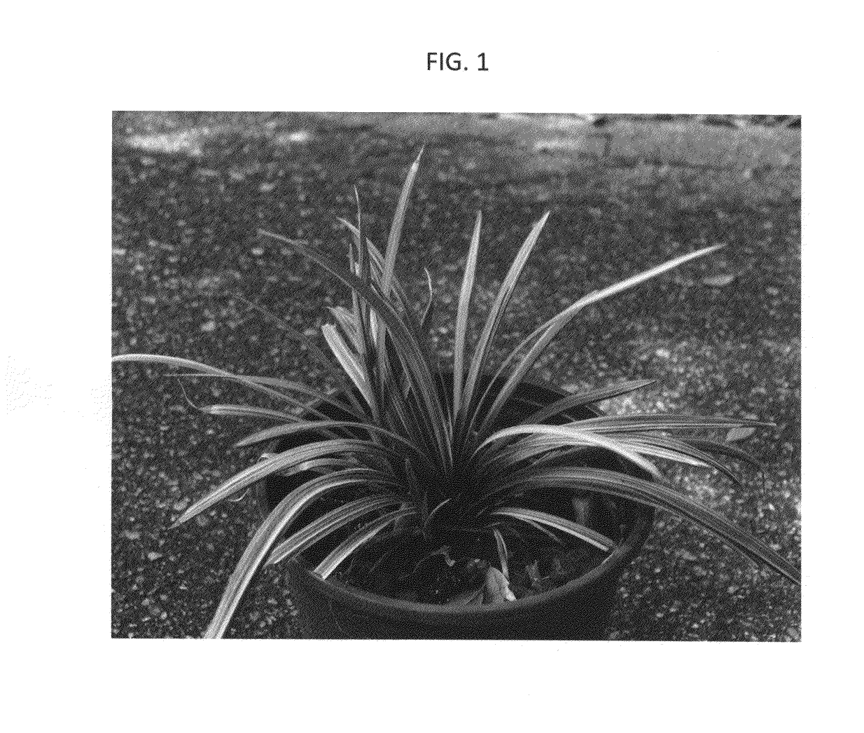

FIG. 2 shows the foliage variegation color and pattern exhibited on the juvenile shoots of ‘VS001’.

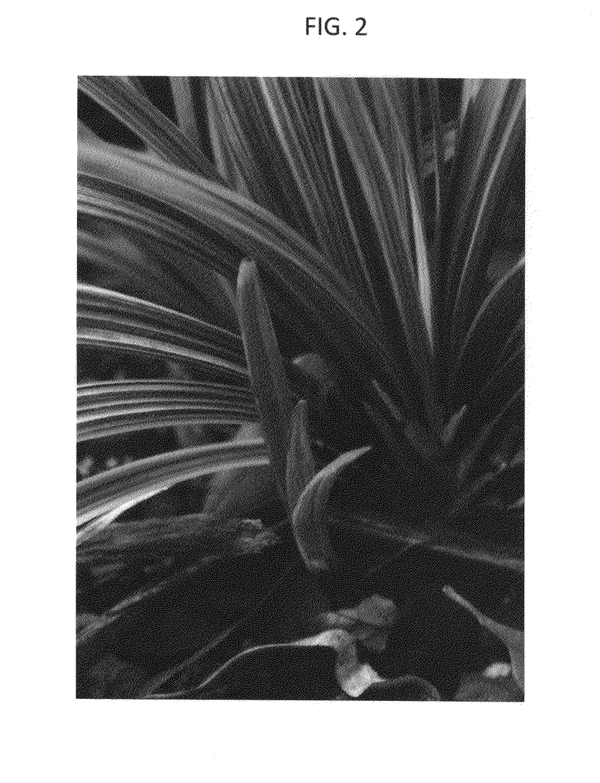

FIG. 3 shows one embodiment of the foliage variegation color and pattern on the mature foliage of ‘VS001’

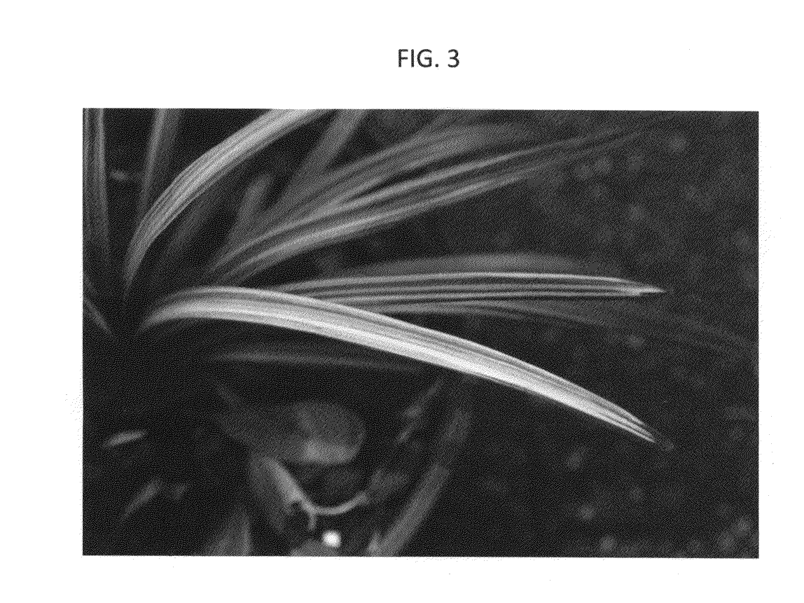

FIG. 4 shows an alternative embodiment of the foliage variegation color and pattern on the mature foliage of ‘VS001’

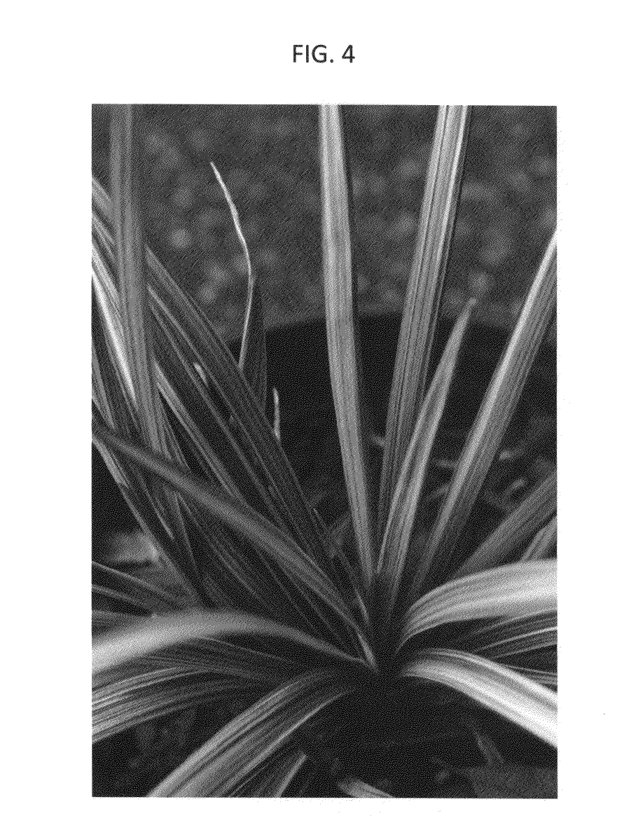

### BOTANICAL DESCRIPTION OF THE PLANT

The following is a detailed botanical description of a new and distinct variety of a Liriope muscari ornamental plant known as ‘VS001’. Plant observations were made on plants grown in Awendaw, S.C. Unless indicated otherwise, the descriptions disclosed herein are based upon observations made in July 2013 of mature ‘VS001’ plants, approximately 8 months of age, grown from rooted cuttings planted in soilless potting media in 15 cm nursery pots. Plants were grown under cover of shade; fertilized with slow release granular fertilizer and regularly watered with overhead irrigation. No pest or disease measures were taken during production.

Those skilled in the art will appreciate that certain characteristics will vary with older or, conversely, younger plants. ‘VS001’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 1986 edition. Note that generic color descriptions such as ‘green’ do not exist in the R.H.S. charts and the corresponding R.H.S. colors are quoted.

‘VS001’ is a selection of Liriope muscari that is characterized by foliage variegation ranging from green to lime-green striations in juvenile shoots and immature foliage, to green and pale yellow to white striations in mature foliage and varying amounts of each color at intermediate stages of leaf development. This feature and other characteristics are apparent from the description provided below.
Growth habit, dimensions and color:
Plant description:
   Plant habit.—Rhizomatous herbaceous perennial with an arching habit and curled leaves.
   Height.—20 cm, as measured.
   Width.—30 cm, as measured.
   Bloom period.—Summer.
   Hardiness.—USDA Zone 6 to 10.
   Environmental tolerances.—Prefers to be grown in partial shade although it is tolerant of full sun. Drought tolerant once established; moderate to good recovery with watering after severe wilting. Tolerates a wide range of soil types from sandy loam to loamy clay.
   Pest and disease susceptibility or resistance.—In common with the species, none of note.
   Propagation.—Propagation is accomplished through division of rhizomes.
   Time to initiate roots.—Approximately 2.5 weeks.
   Crop time.—Depending on latitude of and microclimate of growing location, a fully rooted cutting requires approximately 3 months with an additional 5 to 7 months needed to produce a mature and marketable 15 cm container.
Roots: The roots of ‘VS001’ are fibrous and freely-branched, colored white, fleshy and thick with terminate tubers, similar to other Liriope muscari; high root density. Very short and tightly clumped subsurface rhizomes which root at nodes.
Stem:
   Branching habit.—Acaulescent, rhizomatous plant with shoots emerging from rhizomes with an upright attitude.
Basal shoots:
   Shoots density.—5 divisions, or propagules, per 15 cm nursery pot, with the oldest propagules near the center of the plant's crown possessing 34 leaves and shoots of varying ages and sizes.
   Shoot strength.—Medium.
   Cross section.—Convex.
   Shoot color (adaxial surfaces).—Variegated; comprised of fine parallel striations of yellow-green 145B and green 137C, randomly occurring from striations; fading to a solid yellow-green 145D at and near the base.
   Shoot color (abaxial surfaces).—Variegated; comprised of fine parallel striations of yellow-green 145D and green 138A, randomly occurring from striations; fading to a solid yellow-green 145D at and near the base.
   Shoot surface texture.—Smooth.
Foliage:
   Type.—Evergreen.
   Arrangement.—Basal.
   Division.—Simple.
   Attitude.—Pendulous and curled.
   Shape.—Linear.
   Apex.—Acute.
   Base.—Sheathed.
   Cross section.—Convex.
   Venation.—Parallel.
   Vein color (adaxial surfaces).—indistinguishable from the surrounding foliage.
   Vein color (abaxial surfaces).—indistinguishable from the surrounding foliage.
   Margins.—Entire.
   Attachment.—Acaulescent.
   Texture.—Smooth and glossy.
   Surface hairiness (adaxial surface).—Glabrous.
   Surfaces hairiness (abaxial surface).—Glabrous.
   Mature leaf dimensions.—short and broad; length 215 mm, average width 11 mm at widest point.
   Mature leaf color (adaxial & abaxial surfaces).—Variegated; comprised of parallel striations of varying thickness; color of said striations closest to green 139A and yellow 8C to 8D. Abaxial surface colors are the same, only with higher glaucosity giving the underside a slight sheen; i.e. silvered appearance.
   Petiole.—Leaves are acaulescent; sessile.
   Stipules.—Absent.
Inflorescence: The inflorescence is a raceme consisting of approximately 80 to 120 pedicellate, simple flowers which are freely arranged in axillary fascicles of two to six. Each fascicle is subtended by a small lanceolate-shaped laminar bract, colored yellow-green 144B. Pedicels are 3 to 4 mm long and approximately 1 mm wide. Pedicel color is red-purple 65B. Raceme length ranges from 7 to 9 cm. Peduncle length ranges from 10 to 14 cm and the raceme position is at or slightly above the foliage at anthesis. Peduncle color is yellow-green 144A and transitions to a combination of grey-purple 185C to 186D at the raceme.
Buds: Obovate flower buds are approximately 4 mm in length and 3 mm in width. Color is purple 78D.
Flowers: The flowering season is summer to autumn. Perianth is polypetalous with 6 tepals forming a cupped perianth, with a diameter ranging from 4 to 5 mm at anthesis. Mature tepal color is red-purple 65B. Margins entire. Apex is obtuse or rounded. Anthers are bilobate and small (less than 1 mm long); color is yellow RHS 5C. Stigma is violet RHS 65B. Flowers are persistent and non-fragrant.
Fruit and seeds: Globose berries, approximately 5 mm in diameter, each containing one seed. Berries are glaborous, shiny and have a color that approximates to green 139A when immature and maturing to black 202A late in the season.
Comparison of ‘VS001’ with Other Varieties of Liriope muscari: ‘VS001’ is similar to the parent, ‘samantha’, in most respects with the exception of the presence of foliage variegation ‘VS001’ has foliage variegation colors as herein described while the foliage of the parent is solid green (RHS 147A). When compared with Liriope muscari ‘variegata’, ‘VS001’ has a different foliage variegation pattern and flower color. While the color striations of both are relatively close in color, ‘VS001’ exhibits fine pale-yellow to white parallel striations which occur in a random pattern across the leaf blade, whereas the pale-yellow to white striations of ‘variegate’ are thicker and generally marginal. The flowers of ‘VS001’ exhibit a generally pink appearance, corresponding to red-purple 65B, whereas the flowers of ‘variegate’ are violet 86C. The combination of leaf variegation with thin, randomly-occurring striations and pink flowers makes ‘VS001’ a desirable ornamental plant suited for mass production for container and landscape use.

That which is claimed is:

1. A new and distinct variety of Liriope muscari plant named ‘VS001’, substantially as described and illustrated herein.
